(19) United States
(12) Patent Application Publication
(10) Pub. No.: US 2006/0292199 A1
(43) Pub. Date: Dec. 28, 2006

(54) PMMA bone cement containing antibiotic/antibiotics

(75) Inventors: Klaus-Dieter Kuhn, Marburg (DE); Sebastian Vogt, Erfurt (DE)

Correspondence Address: Kurt G. Briscoe;Norris, McLaughlin & Marcus P.A., 18th Floor, 875 Third Avenue, New York, NY, 10022, US

(73) Assignee: HERAEUS KULZER GMBH, Hanau (DE)

(21) Appl. No.: 11/234,034

(22) Filed: Sep. 23, 2005

(30) Foreign Application Priority Data
Oct. 7, 2004 (DE) 10 2004 049 121.6

(51) Int. Cl. A61K 38/14; A61K 31/7034; A61K 31/545; A61K 31/496; A61K 9/14; A61F 13/00

(52) U.S. Cl. 424/422; 424/489; 514/35; 514/200; 514/8; 514/253.08

(57) ABSTRACT

A PMMA bone cement containing an antibiotic/antibiotics is described which is characterized in that, in the powder component, 0.1-5.0% by weight of water soluble, glasstype antibiotic/antibiotics granules with a particle diameter in the region of 50-1000 μm are contained which are built up of glass-type antibiotic/antibiotics primary particles bonded to each other which have a particle diameter in the region of 1-70 μm.

The subject matter of the invention is a PMMA bone cement containing antibiotic/antibiotics, with a powder component and a liquid component.

PMMA bone cements (polymethylmethacylate bone cements) containing antibiotics have been known since the sixties of the 20th century on the basis of work by H. W. Buchholz and the commercial company Kulzer (W. Ege, K.-D. Kühn: industrial development of bone cement—25 years of experience. In: bone Cement and Cementing Technique. Eds. G. H. I. M. Walenkamp, D. W. Murray, Springer Verlag Heidelberg 2001, in press: H. W. Buchholz, E. Engelbrecht: Über die Depotwirkung einiger Antibiotika beim Vermischen mit dem Kunstharz Palacos (Concerning the depot effect of some antibiotics on mixing with the synthetic resin Palacos), Chirurg 41 (1970) 511-515). These PMMA cements have found wide acceptance and are used on a large scale for fixing endoprotheses (K.-D. Kühn: Knochenzemente für die Endoprothetik: ein aktueller Vergleich der physikalischen and chemischen Eigenschaften handelsülicher PMMA-Zemente (Bone cements for endoprothetics: an up-to-date comparison of the physical and chemical properties of commercial PMMA cements), Springer-Verlag Berlin Heidelberg New York, 2001). The antibiotic integrated into the PMMA bone cement is released more or less rapidly locally after implantation at the bone cement/bone interface and is intended to prevent the bacterial colonization there. The aim is as high an initial release as possible such that the minimum bactericidal concentration (MBC) of the antibiotic used vis-á-vis the clinically relevant germs is achieved safely and exceeded at the bone cement/bone interface. The antibiotic most frequently used in PMMA bone cements so far has been the broadly effective gentamicin.

The invention is based on the task of developing a PMMA bone cement which exhibits a very high initial antibiotic/antibiotics release. The antibiotic is to be released in large quantities from the bone cement within the first 24 hours following curing of the bone cement.

The task is achieved by way of the subject matter described hereinbelow. The PMMA bone cement is characterized in that, in the powder component, 0.1-5.0% by weight of water-soluble, glass-type antibiotic/antibiotics granules with a particle diameter in the region of 50-1000 μm, preferably 63-900 μm, are contained which are built up of glass-type antibiotic/antibiotics primary particles bonded to each other which have a particle diameter in the region of 1-70 μm.

The powder component of the PMMA bone cement should be understood to be a mixture of at least one polymethylmethacylate in powder form or a copolymer which is built up of methylmethacrylate and methylacrylate, an x-ray opaquer in powder form such as zirconium dioxide and/or barium sulphate and a radical initiator such as dibenzoyl peroxide. If necessary, the constituents of the powder component are dyed with a pharmaceutically acceptable dye. After mixing with the liquid component which is built up of methylmethacrylate (MMA) in which a radical activator such as N, N-dimethyl-p-toluidine is dissolved, the powder component gives a plastically deformable paste which is cured independently after a few minutes by the on-setting radical polymerization of the methylethylacrylate.

The term glass-type antibiotic/antibiotics granules should be understood to mean granules of one or several antibiotics which do not exhibit any crystalline structure recognizable under the light microscope and appear to be transparent and/or opaque. The antibiotic/antibiotics granules have a glass-type appearance. Moreover, the antibiotic/antibiotics granules have a particle diameter of approximately 50-1000 μm and are built up of glass-type antibiotic/antibiotics primary particles which are firmly bonded to each other. The term firmly bonded glass-type antibiotic/antibiotics primary particles should be understood to mean that the granules built up of primary particles bonded to each other are so stable that these can be ground without problems together with x-ray opaquers with an abrasive effect of the powder component, such as zirconium dioxide and barium sulphate or mixed with suitable devices without a significant decomposition of the granules into the primary particles taking place. Glass-type means in this connection also that no crystals are recognizable under the light microscope in the primary particles and that the primary particles themselves do not represent crystals. Moreover, the term glass-type means that the primary particles appear to be transparent and/or opaque.

The PMMA bone cement produced according to the invention exhibited a very high antibiotics release under in vitro conditions at 37° C.

It is advantageous that the particle boundaries of the glass-type primary particles are recognizable under the light microscope only at the surface of the antibiotic/antibiotics granules. This means that it is possible to draw approximate conclusions from the surface properties of the granules under the light microscope on the size of the antibiotic/antibiotics primary particles.

It is appropriate that the antibiotic/antibiotics granules comprise at least one representative from at least one of the groups of the aminoglycoside antibiotics, the lincosamide antibiotics, the fluoroquinolone antibiotics, the glycopeptide antibiotics and the nitroimidazols. The antimicrobially effective chemotherapeutics from the group of the nitroimidazols are, in a simplified manner, also understood to be antibiotics. These chemotherapeutics have a mainly bactericidal effect against anaerobic germs.

It is appropriate for the antibiotic/antibiotics granules to comprise preferably gentamicin sulphate, gentamicin hydrochloride, amikacin sulphate, amikacin hydrochloride, tobramycin sulphate, tobramycin hydrochloride, clindamycin hydrochloride, lincosamine hydrochloride, moxifloxacin, ciprofloxacin, telcoplanin, vancomycin, ramoplanin, metronidazol, tinidazol or omidazol or their mixtures. Apart from these water-soluble antibiotic salts and antibiotics, salt forms of the antibiotics with a low solubility in water such as palmitates, myristates and laureates may be integrated additionally into the antibiotic/antibiotics particles. In addition, it is also possible for antibiotics from the group of oxazolidones such as linezolid to be integrated into the granules.

It is, moreover, advantageous for the antibiotic/antibiotics granules to additionally contain, if necessary, polyvinylpyrrolidone and/or polyethylene glycol and/or polyethylene oxide and/or maltose and/or sorbitol and/or mannitol as auxiliary agents. By means of these auxiliary agents, the antibiotic/antibiotics granules can be stabilized. It is also within the framework of the invention that the antibiotic/antibiotics granules are stabilized by other toxicologically acceptable polymers such as gelatine, collagen and dextran. In a further sense of the invention, it is possible to derive from the antibiotic/antibiotics granules according to the invention those granules which are formed from antibiotic/antibiotics crystals which have been bonded or glued together with adhesive auxiliary agents to form antibiotic/antibiotics granules with particle sizes in the region of 50-1000 μm, preferably 63-900 μm.

The invention will be explained by way of two examples without, however, limiting the invention.

### EXAMPLE 1

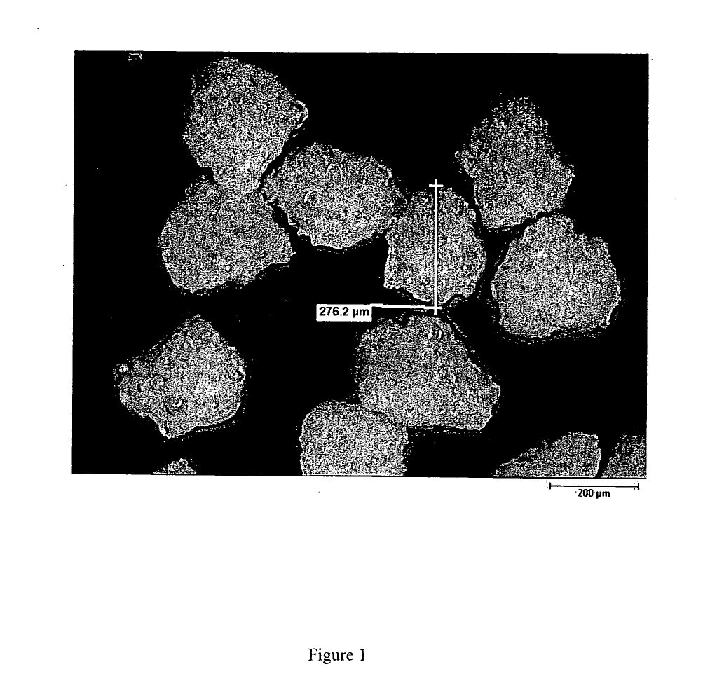

In FIG. 1, typical antibiotic granules of gentamicin sulphate according to the invention with a sieve fraction of 125-250 μm are shown, the primary particles being clearly recognizable by the surface structure.

### EXAMPLE 2

To test the PMMA bone cement according to the invention, release investigations were carried out on sample bodies. The preparation of the sample bodies was carried out in such a way that 40.0 g of the powder component of the bone cement Palacos® (Heraeus Kulzer) in each case were mixed with

Variant a) 0.8 g gentamicin sulphate with a sieve fraction of <63 μm

Variant b) 0.8 g of the glass-type gentamicin sulphate granules built up from primary particles with a sieve fraction of 63-250 μm.

Subsequently, these modified powder components were mixed with 20.0 g of the monomer component each. A green paste was formed which was spread into hollow forms and cured therein after a few minutes. The cylinder-shaped sample bodies thus formed had a height of 1 cm and a diameter of 2.5 cm. 5 sample bodies per cement variant were produced in each case. The sample bodies were stored separately in 20 ml of distilled water at 37° C. The release medium was completely removed daily and the quantity of gentamicin released therein was determined. The sample bodies were then stored then stored again in 20 ml of fresh distilled water each at 37° C. The determination of the released gentamicin was carried out with a TDX analyzer from Abott. The mass of gentamicin base released in each case was indicated per gram of sample body in the following table as a function of the storage time of the sample bodies in the release medium.

| | Mass of gentamicin base released (μg/g) | | | |
|---|---|---|---|---|
| Storage time | 1 | 2 | 3 | 4 |
| [d] | | | | |
| Variant a) | 113 | 6 | 4 | 0 |
| Variant b) | 217 | 33 | 17 | 11 |

What is claimed is:

1. PMMA bone cement comprising antibiotic/antibiotics, said PMMA bone cement further comprising a powder component and a liquid component, wherein the powder component comprises 0.1-5.0% by weight of water-soluble, glass-type antibiotic/antibiotics granules of glass-type antibiotic/antibiotics primary particles bonded to each other that have a particle diameter in a range of 1-70 μm.

2. PMMA cement according to claim 1, wherein the antibiotic/antibiotics granules have particle diameters in the range of 50-1000 μm.

3. PMMA bone cement according to claim 1, wherein particle limits of the glass-type antibiotic/antibiotics primary particles are recognizable by light microscopy only at a surface of the antibiotic/antibiotics granules.

4. PMMA bone cement according to claim 1, wherein the antibiotic/antibiotics granules comprise at least one representative selected from the group consisting of aminoglycoside antibiotics, lincosamide antibiotics, fluoroquinolone antibiotics, glycopeptide antibiotics or nitroimidazols.

5. PMMA bone cement according to claim 1, wherein the antibiotic/antibiotics granules comprise gentamicin sulphate, gentamicin hydrochloride, amikacin sulphate, amikacin hydrochloride, tobramycin sulphate, tobramycin hydrochloride, clindamycin hydrochloride, lincosamine hydrochloride, moxifloxacin, ciprofloxacin, telcoplanin, vancomycin, ramoplanin, metronidazol, tinidazol or omidazol or a mixture thereof.

6. PMMA bone cement according to claim 1, wherein the antibiotic/antibiotics granules additionally comprise polyvinyl pyrrolidone and/or polyethylene glycol and/or polyethylene oxide and/or maltose and/or sorbitol and/or mannitol as auxiliary agents.

7. A method of joining bone segments or for attaching an artificial implant to bone, said method comprising cementing the bone segments or the artificial implant and bone together with a PMMA bone cement according to claim 1.
